(19) United States
(12) Patent Application Publication
(10) Pub. No.: US 2021/0369646 A1
(43) Pub. Date: Dec. 2, 2021

(54) Composition for local tumescent anesthesia

(71) Applicant: Maria del Pilar Reyes Agüero, Pharr, TX (US)

(72) Inventor: Maria del Pilar Reyes Agüero, Pharr, TX (US)

(21) Appl. No.: 16/922,500

(22) Filed: Jul. 7, 2020

(30) Foreign Application Priority Data
May 29, 2020 (MX) MX/A/2020/005625

(51) Int. Cl. A61K 31/137; A61K 31/445; A61K 31/195; A61K 47/02; A61P 23/02

(52) U.S. Cl. CPC A61K 31/137; A61K 31/445; A61P 23/02; A61K 47/02; A61K 31/195

(57) ABSTRACT

The present invention provides a composition for local tumescent anesthesia wherein 100 cc volume of the composition comprising 0.5-1 ml of local vasoconstrictor agent of concentration 1 mg/ml, 20-40 ml of local anesthetic agent of concentration 7.5 mg/ml; 1-2 ml of anti-fibrinolytic agent of concentration 500 mg/5 ml. The composition together generate less pain, minimize the bleeding risks and its post-operative complications, promote the tissue dissection and exerts a longer anesthetic effect. The local tumescent anesthesia composition is diluted in saline or ringer lactate solution. The composition may be employed in various surgeries selected from cosmetic surgery, plastic surgery, reconstructive surgery, head and neck surgery, ENT surgery, gynecological surgery, orthopedic surgery, proctological surgery.

### CROSS REFERENCE TO RELATED APPLICATION

This application claims the benefit of and takes priority from Mexican Patent Application Serial No. MX/a/2020/005625 filed on May 29, 2020, the contents of which are herein incorporated by reference.

### TECHNICAL FIELD

The embodiments herein generally relates to cosmetic surgery and anesthesiology and more particularly, relates to a composition in the liquid solution form which generate less pain, minimize the bleeding risks and its post-operative complications, promote the tissue dissection and exerts a longer anesthetic effect.

### BACKGROUND OF THE INVENTION

Nowadays, it becomes a serious concern for the physicians and surgeons to cope up with the bleeding episodes occurring while performing traumatic and cosmetic surgeries. The problems related to bleeding poses a major threat during surgeries and may cause severe blood loss in patient undergoing surgical operations. It may lead to various surgical complications and causes severe abnormalities in the clotting mechanism of the body. Although high mortality rates are witnessed for surgical procedure, and it may increase while operating due to severe bleeding problems. Approximately 75% to 90% of intraoperative and early postoperative bleeding occurs due to the loss of blood clotting factors. The uncontrolled bleeding leads to a combination of troublesome consequences which in turn worsen the clotting process, further aggravates the problem in a blood regulation and hemostasis mechanism. At present, the medical science needs a more sophisticated and comprehensive medical procedures to tackle the excessive bleeding problems discussed herein.

Hereinafter, it becomes a prime necessity to acquire a technique which is having appreciable command over excessive bleeding issues and complications. Tumescent anesthesia is a surgical technique used in cosmetic and dermatologic procedures involving subcutaneous infiltration with highly diluted lidocaine and epinephrine. It has transformed the surgical science and lipoplasty from bloody surgeries to a painless and bloodless procedure. It was originally devised for use in liposuction, but has since been applied to other surgical situations, including plastic surgery, burn care and vascular surgery. It is relatively safe way to achieve extensive dermal anesthesia of skin by safeguarding systemic toxicity. The subcutaneous infiltration of a large volume of very dilute lidocaine and epinephrine causes the targeted tissue swollen and tumescent and permits other painful procedures to be performed on patients without subjecting them to inherent risks of general anesthesia, and with reduced blood loss due to the vasoconstriction induced by epinephrine.

Moreover, tumescent local anesthesia (TLA) has plenty of applications in Dermasurgery and plastic surgery of human skin anatomy. TLA has been exhaustively utilized by medical science for aesthetic and anesthetic procedures which inherently determined its effectiveness in past. Nowadays it becomes a trend and a first choice of many patients affected by acute skin related medical complications. TLA is majorly practiced in the field of Dermasurgery such as liposuction, fat transfer, dermabrasion, hair transplant, axillary hyperhidrosis, laser resurfacing, flaps and grafts, gynecomastia, ambulatory phlebectomy, saphenous vein closure etc. It has been predominantly exercised in non-derma surgery related field as well which speaks about its vast abundant amount of applications in surgical science. Tumescent local anesthesia (TLA) can also be useful in abdominoplasty, breast surgery, mastectomy, burn surgery, facelift, craniofacial surgery, sentinel lymph node biopsy, stem cell harvesting, pressure ulcer closure, proctology, inguinal herniography, large cutaneous reactions etc. Earlier it has been demonstrated that the tumescent local anesthesia technique has shown excellent activity in reducing blood loss and transfusion requirement in flap breast reconstruction as well. No adverse effects on breast mound or abdominal donor-site has been observed.

In “Carlson G W. Breast J. 2005, 11; 100-2”, the tumescent technique was performed using local anesthesia for mastectomy surgical procedure. It further mentions about tumescent technique of infiltrating dilute lidocaine with epinephrine (25 ml of 1% lidocaine and 1 ml of 1:1000 epinephrine) via an infusion pump. However it has evidenced in severe complications but caused no morbidity. Conclusively, it has proven to be mildly safe and effective for total Mastectomy in patients but not safe in candidates for general anesthesia. Tumescent local anesthesia produces a comparatively long lasting effect because of its extremely slow systemic absorption. For its proper administration, it requires skilled infiltration and a good knowledge of pharmacology. Due to the lidocaine as the primary component of the composition, it shows mild toxic effects when absorbed into the systemic circulation and increased morbidity.

In the cited art titled “Fast track concepts in total knee arthroplasty” by Petersen W. et al. which discloses surgical technique having preoperative administration of 1 g tranexamic acid, thereafter intraoperative local infiltration anesthesia was carried out in patients. After femoral and tibial bone resection and before cementing the femoral and tibial components, approximately 40 ml of ropivacaine is injected into the posterior capsule. This is followed by injection to the medial and lateral collateral ligaments with approximately 20 ml each and infiltration of the extensor tissue also with approximately 20 ml local anesthetic. After cementing, the subcutaneous tissue is infiltrated with approximately 50 ml ropivacaine solution. As a result, it was observed that the patient received preoperatively 1 g tranexamic acid and intra-articular infiltration anesthesia shown mild algesia and painful symptoms, which is proved to be concomitantly inefficacious.

In another cited art titled “Ambulatory unicompartmental knee arthroplasty” by N. Ruiz X et al. which relates to the study of using a fast-track strategy enabling an outpatient UKA (Unicompartmental knee arthroplasty) procedure that is both feasible and safe, for the large majority of selected patients. Furthermore, it does not affect patient satisfaction. Preoperatively the patient received the solution of dexamethasone 2 mg/10 kg, tranexamic acid 2 g and cefazoline 2 g and postoperatively the patient received a capsular injection of solution of Ropivacaine (150 mg)+Bi-Profenid (30 mg). This work has several limitations outlined in the current discussion. Main one being that there was no control groups so that we do not assess the potential for benefit of the patients. The series is limited to 50 outpatient UKAs over 2 years. Since the end of the study, 32 more outpatient UKAs in 2016 were operated on using the same protocol with identical results. The data were only collected during the first postoperative month, which may exclude any general or local complications that occurred later on. There were no comparisons with other anesthetic techniques (SA, RA) conclusively.

In one of the cited art, U.S. '8,549A1 which discloses a method of administering anesthesia to a patient prior to and during a variety of cosmetic surgical procedures. More specifically, the invention relates to techniques and procedures for a patient to undergo cosmetic surgical procedures, including tumescent liposuction, without requiring the use of general anesthesia or intravenous (IV) administered anesthesia, and to provide a more pleasant experience that reduces the anxiety of a patient prior to and while undergoing surgery, as well as provides optimal results post-surgery. Anesthetizing solution comprises lidocaine, epinephrine and sodium bicarbonate; and infiltrating the anesthetizing solution in a targeted area of the patient. But herein as mentioned, the cited prior art has shown higher arrhythmogenic and cardiotoxicity effect with lower absorption capability and elevated morbidity in rats.

In the cited art, titled “Tumescent Anesthesia: Lidocaine Dosing Dichotomy” (RUDOLPH H. de JONG) which discloses tumescent anesthesia with highly diluted lidocaine and epinephrine has revolutionized the lipoplasty technique from a painful to the painless and bloodless procedures. Lipoplasty defined as surgical resection of unwanted body fat under direct vision, has long been the only option for removing fat deposits. The administration of diluted lidocaine has transformed the subdermal drug supply; it has been postulated to buffer up to 1 mg of lidocaine per gram of body tissue. Certain limitations apply when using high-dose lidocaine for tumescent anesthesia, proper dilution of lidocaine to 0.1% or less, and adding 1 mg fresh epinephrine to each liter are of the more efficacious value. The total lidocaine delivery in surgical tissue decreases the liver's clearance capacity by cytochrome P450 inhibition; functional impairment demands a corresponding reduction in the total lidocaine concentration. When combining tumescent lidocaine with full-strength lidocaine to anesthetize more densely innervated structures such as breast or face, optimum drug dosing must be maintained because of the more potential of lidocaine to cause systemic toxicity.

In another cited art, Daniel S. Behroozan et. al. which relates to the use of dermal tumescent anesthesia in cutaneous surgical procedure. The dermal tumescent anesthesia produces superior local anesthesia by directly injecting larger amounts of diluted anesthetic solution into dermis, and also a reduced amount of bleeding intraoperatively and postoperatively. The composition is used in combination as 0.5% lidocaine with epinephrine 1:200,000 (xylocaine) that is buffered with 8.4% sodium bicarbonate. The use of dermal tumescent anesthesia results in lesser amounts of anesthetic solution injected into the dermis compared with when injecting directly into fat to obtain adequate anesthesia.

In the cited art, U.S. '3,024B2 which discloses a method of subcutaneous delivery of drug or a therapeutic agent to a subject comprising administering to said subject a tumescent composition comprising: a local tumescent solution wherein the anesthetic solution having the concentration of the lidocaine approximately 100 mg to 1500 mg per Liter of solution and the antiviral agent having concentration of the acyclovir is 0.1-10 g/L and the vasoconstrictor having the concentration of epinephrine is approximately 0.2 to 1.5 mg/L. It is pertinent to mention that the present invention is not recommendable due to its high systemic toxicity and inability to avoid high bleeding risks and slower absorption into systemic circulation.

In another cited art, by Jeffery Klein, Klein's solution is used as local tumescent anesthesia in which it comprises the following components: Lidocaine (0.5 gm to 1 gm/Liter), Epinephrine (0.5 mg to 1 mg/Liter), and Sodium Bicarbonate with 10 meq/Liter having the pH approx. 7. Meanwhile, it is witnessed that lidocaine which is the primary component, exerted a higher arrhythmogenic effect with slower adsorption rate as well as high cardiotoxicity. In the Klein's solution the epinephrine acts as a potent α and β-adrenergic agonist at the principal site of action on the small arterioles and precapillary sphincters. Epinephrine herein serves the role of a vasoconstrictor and shows excellent vasoconstriction activity on capillaries. In contrast, it is a demerit of the present composition due to its failure in exerting a longer anesthetic effect as well as blood retention capacity.

Hence, looking at the problems in the prior art, there is a significant need to develop a local tumescent anesthesia which could generate less pain, minimize the bleeding risks and its post-operative complications, promote the tissue dissection and exerts a longer anesthetic effect.

### SUMMARY OF THE INVENTION

The invention overcomes the aforementioned problems by introducing local tumescent anesthesia composition. It can exert prolonged anesthetic effects and the vessels contraction minimizing bleeding risks and its postoperative complications that may lengthen the operation theatre stay while operating the patients.

In the most preferred embodiment of the present invention, a composition for local tumescent anesthesia wherein 100 cc volume of the composition comprising 0.5-1 ml of local vasoconstrictor agent of concentration 1 mg/ml, 20-40 ml of local anesthetic agent of concentration 7.5 mg/ml and 1-2 ml of anti-fibrinolytic agent of concentration 500 mg/5 ml. The local vasoconstrictor agent is epinephrine, local anesthetic agent is preferably Ropivacaine, anti-fibrinlolytic agent is preferably tranexamic acid. The various components present in the composition together generate less pain, minimize the bleeding risks and its post-operative complications, promote the tissue dissection and exerts a longer anesthetic effect.

In another embodiment of the invention, all the components disclosed are FDA (Food and Drug Administration) approved, safe for patient compliance and acceptable for various medical purposes. The composition prevents the complications of bleeding after surgery and postoperative conditions as well as to ensure the suitability of the tissues. It can achieve a prolonged duration of anesthesia alongside a persistent effect for successfully anesthetizing a patient with no harmful effects. It is highly recommended as it minimizes bleeding and other postoperative complications. Further, in the said composition, epinephrine is in the form of a racemic epinephrine. It is pertinent to give a more emphasis on the fact that, Ropivacaine is preferred over other local anesthetics due to its lower cytotoxicity and arrhythmogenic effects.

In another preferred embodiment, the local anesthetic composition is present in the form of a liquid solution which is made in saline solution or ringer lactate solution. While performing the surgical procedure, the liquid solution facilitates in numbness of the body tissue undergoing surgery thereby minimizing the excessive bleeding risk.

These and other aspects of the embodiments herein will be better appreciated and understood when considered in conjunction with the following description. It should be understood, however, that the following descriptions, while indicating preferred embodiments and numerous specific details thereof, are given by way of illustration and not by way of limitation. Many changes and modifications may be made within the scope of the embodiments herein without departing from the spirit thereof, and the embodiments herein include all such modifications.

### DETAILED DESCRIPTION OF PREFERRED EMBODIMENTS

The embodiments herein and the various features and advantageous details thereof are explained more fully with reference to the non-limiting embodiments that are illustrated and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

According to the present invention, it is contemplated that the composition (Reye's solution) for local tumescent anesthesia consists of combination of different components prepared in the form of liquid solution is very effective in minimizing bleeding risks. The one of the component epinephrine prominently known as adrenaline provides the local vasoconstriction effect at various sites of the infiltration. The composition includes Ropivacaine which demonstrates its function as a local anesthetic agent concomitantly provides a prolonged anesthetic and numbing effect. Another most crucial ingredient of the invention is tranexamic acid, a drug which is classified in the category of anti-fibrinolytic agents. Tranexamic acid is a substance which significantly reduces post-operative bleeding by altering selectively of the fibrinolytic system; it inhibits the formation of plasmin temporarily preventing the dissolution and degradation of fibrin clots.

In the present invention, the composition may be useful and employed in various surgeries according to this invention most preferably in cosmetic surgery, plastic surgery, reconstructive surgery, head and neck surgery, ENT surgery, gynecological surgery, orthopedic surgery, proctological surgery.

Furthermore, obtained experimental data evidence about the ability of the current composition named as Reye's solution achieves greater efficacy in reducing the bleeding episodes and its post-treatment complications with sustainable anesthetic effects. The available experimental data speaks for the surprising efficacy with usefulness in medical anesthetic treatments of the local tumescent anesthesia composition. Any references to the current composition or Reye's solution in this description should be understood as liquid solution form for its administration.

In the present invention, the composition for local tumescent anesthesia comprising epinephrine, Ropivacaine and tranexamic acid can be used in pharmaceutical preparations in combination with a pharmaceutically acceptable carrier. The components may be dissolved in the pharmaceutically acceptable carriers selected, for example 0.9% Saline Solution (100 ml) and Hartmann (Ringer's lactate) solution (100 ml). The term “carrier” applied to pharmaceutical compositions of the invention refers to an excipient or vehicle with which active components are administered. The vehicle as a carrier used must be sterile in nature to prevent the composition from microbial degradation. The composition can be employed with a 3 cc syringe and 30 G needle to get the subcutaneous layer in the tissue, it also can be injected as well in dermis; stop injection and pull back in case of bleed stain into the needle; it is forbidden to inject into the bloodstream.

Use of the composition of the present invention in the manufacture of a surgical medicament for significantly reducing bleeding risks, in usual manner comprising the step of admixing an effective amount of different components of the invention with a pharmaceutically-acceptable sterile vehicle or carrier. Reyes's solution is a mixture of elementary compounds used separately in different surgeries: cosmetical, gynecological, orthopaedical and so on, that using it together generate less pain, minimize the bleeding risks and promote the tissue dissection, leading to a safety surgery.

As disclosed herein, the dose of the components in the compositions of the present invention is determined to ensure that the amount administered continuously or intermittently will not exceed an amount determined after considering the individual conditions of a patient.

The composition is provided in the form of parenteral formulation which can be injected into the tissue imparts an excellent and appreciable behavior when administered into the body. A highly potent local anesthetic agent i.e. Ropivacaine, it is having long duration of activity and is supplied in injectable form. It is classified in the category of an amino amide, whose highly lipophilic; it has a middle lifetime from 14 min to 4 hours, it bounds to alpha 1 acid glycoprotein almost entirely with just a 6% free protein form, with an absorption and conjugation in the liver (aromatic hydroxylation transformed into 3-hydroxy-ropivacaine the 99% of all anesthetic agent) and it is excreted through the kidneys into the urine (86% approximately).

Subsequently in another embodiment, Tranexamic acid is used as an anti-fibrinolytic agent for decades for curing bleeding related complications. It has a high affinity to plasminogen and the subsequent form of plasmin reducing the effect on fibrin; after the administration it has a middle lifetime of 3 hours, it has a plasmatic protein bound of 3%, and has no free protein form. It will be excreted through the kidneys into the urine without a conjugated form (90% by the first 24 hours).

Moreover, Epinephrine which is prominently regarded as local vasoconstrictor agent is classified as a sympathomimetic amine, with a vasoconstrictor effect on alpha and beta receptors. The maximum concentration of epinephrine after a subcutaneous injection will be 20-40 minutes. It has a very short middle lifetime of 20 seconds. It will be metabolized by two enzymes, the catechol-o-methyltransferase (extra neural), and monoaminoxidase (intra neural) generating vanillylmandelic acid mostly in the liver. The principal metabolites are conjugated metanephrine (40%), vanillylmandelic acid (40%), dihydroxymandelic acid, metoxihydroxyphenylglycol and unadulterated epinephrine. The 50% of total doses will be excreted by the first 6 hours, the other 50% after next 18 hours into the urine.

### Exemplary Formulations

The following exemplary formulations are given by way of illustration only and are not to be construed as limiting this invention in any way.

Formulation 1: In order to prepare Reye's Solution it combines the following components:
1 ml Epinephrine (Racemic) 1 mg/ml
40 ml Ropivacaine 7.5 mg/ml
1 ml Tranexamic Acid 500 mg/5 ml
Saline Solution 0.9% q.s. 100 ml
   Formulation 2: In order to prepare Reyes Solution it combines the following components:
1 ml Epinephrine (Racemic) 1 mg/ml
40 ml Ropivacaine 7.5 mg/ml
1 ml Tranexamic Acid 500 mg/5 ml
Hartmann (Ringer's lactate) solution q.s. 100 ml

As mentioned, there remains the foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A composition for local tumescent anesthesia, wherein 100 cc volume of the composition comprising:
0.5-1 ml of local vasoconstrictor agent of concentration 1 mg/ml.
20-40 ml of local anesthetic agent of concentration 7.5 mg/ml
1-2 ml of anti-fibrinolytic agent of concentration 500 mg/5 ml;
wherein the composition together generate less pain, minimize the bleeding risks and its post-operative complications, promote the tissue dissection and exerts a longer anesthesic effect.

2. The composition for local tumescent anesthesia as claimed in claim 1, wherein said local vasoconstrictor agent is Epinephrine.

3. The composition for local tumescent anesthesia as claimed in claim 1, wherein said local anesthetic agent is Ropivacaine.

4. The composition for local tumescent anesthesia as claimed in claim 1, wherein said anti-fibrinolytic agent is Tranexamic acid.

5. The composition for local tumescent anesthesia as claimed in claim 1, wherein said composition is in the form of a liquid solution which is made in saline solution or ringer lactate solution.

6. The composition for local tumescent anesthesia as claimed in claim 1, wherein said Epinephrine is a racemic epinephrine.

7. The composition for local tumescent anesthesia as claimed in claim 1, wherein Ropivacaine has lower cytotoxicity and arrhythmogenic effects.

8. The composition for local tumescent anesthesia as claimed in claim 1, wherein said Tranexamic acid decreases the amount of bleeding.

9. The composition for local tumescent anesthesia as claimed in claim 1, wherein the composition can be employed in various surgeries selected from cosmetic surgery, plastic surgery, reconstructive surgery, head and neck surgery, ENT surgery, gynecological surgery, orthopedic surgery, proctological surgery.
